(19) United States
(12) Patent Application Publication
(10) Pub. No.: US 2009/0275580 A1
(43) Pub. Date: Nov. 5, 2009

(54) 3-SPIROINDOLIN-2-ONE DERIVATIVES, PREPARATION AND THERAPEUTIC USE THEREOF

(75) Inventors: Denis BRASSEUR, Chilly Mazarin (FR); Claudine SERRADEIL-LE GAL, Escalquens (FR); Gareth SHACKLETON, White Bay (GB)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/504,278

(22) Filed: Jul. 16, 2009

Related U.S. Application Data
(63) Continuation of application No. 11/682,988, filed on Mar. 7, 2007, now abandoned.
Application No. 11/682,988 is a continuation of application No. PCT/FR2005/002220, filed on Sep. 7, 2005.

(30) Foreign Application Priority Data
Sep. 9, 2004 (FR) 0451998

(51) Int. Cl. A61K 31/5377; C07D 413/12; A61P 25/00; A61P 9/00; A61P 27/02

(52) U.S. Cl. 514/235.2; 544/70

(57) ABSTRACT

The subject of the present invention is a compound of formula (I):

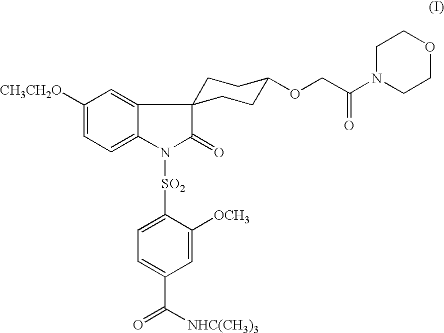

in the base, hydrate or solvate state, in the form of cis/trans isomers or of mixtures thereof, preparation and therapeutic use thereof.

The present invention relates to 3-spiroindolin-2-one derivatives, to the process for preparing them and to the therapeutic use thereof.

A subject of the present invention is compounds of formula (I):

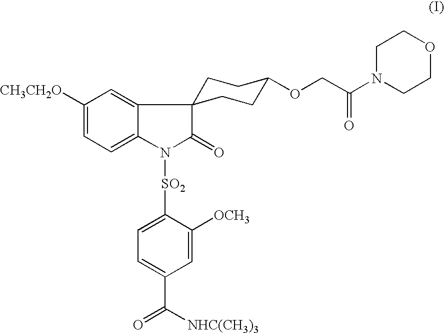

in the base, hydrate or solvate state, in the form of cis/trans isomers or of mixtures thereof.

The compounds of formula (I) contain one or more rings. They can therefore exist in the form of cis/trans isomers. These isomers and mixtures thereof are part of the invention.

The compounds of formula (I) can also exist in hydrate and/or solvate form, i.e. in the form of an association or of a combination with one or more molecules of water or with a solvent. Such hydrates and solvates are also part of the invention.

In accordance with the invention, a compound of formula (I) can be prepared according to the process illustrated by scheme 1 which follows.

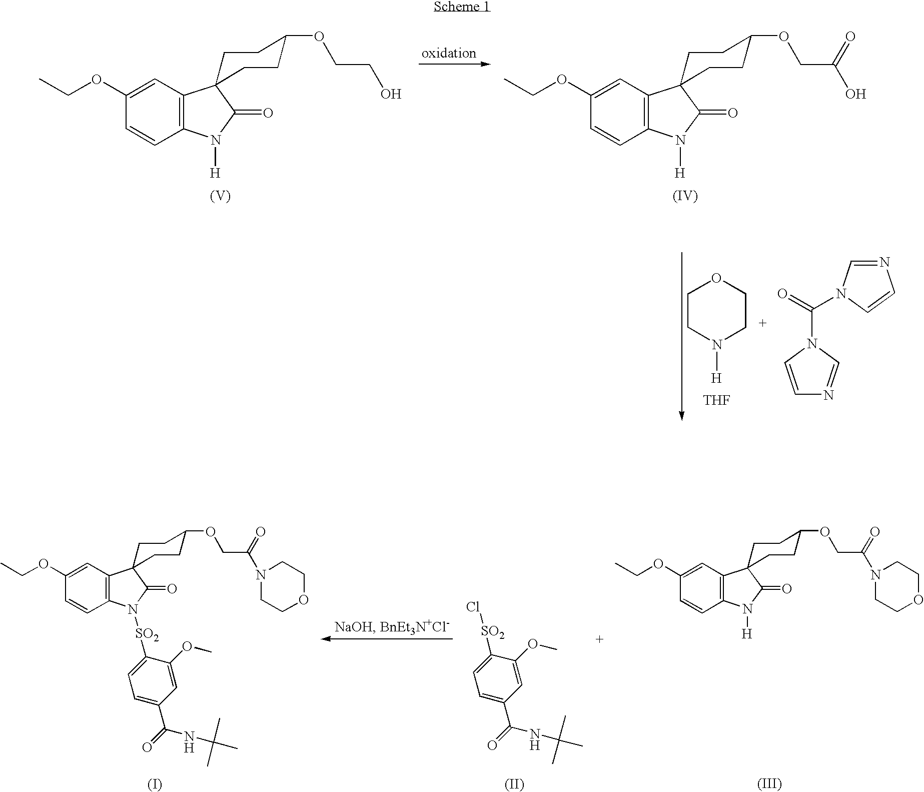

The compound of formula (IV) is obtained by means of an oxidation reaction of the compound of formula (V) using a powerful oxidizing agent such as potassium permanganate or chromium trioxide in the presence of a polar aprotic solvent such as acetone or dimethylformamide. The synthesis of the compound of formula (V) is described in document EP 0 873 309 (preparation 10, compound (III′.1)). Carbodiimidazole and morpholine are added to the compound of formula (IV) obtained above, in the presence of tetrahydrofuran (THF), dichloromethane (CH₂Cl₂) or chloroform, so as to obtain the compound of formula (III). The compound of formula (I) is obtained by adding the compound of formula (III) obtained above to the compound of formula (II), the synthesis of which is described in document EP 0 873 309 (preparation 13, reactant (2).2). The addition of the compound of formula (III) to the compound of formula (II) is carried out in a basic medium in an aprotic solvent such as CH₂Cl₂, THF, dimethylformamide or dimethyl sulfoxide.

When the method of preparing the reactants is not described, they are commercially available or are described in the literature, or else they can be prepared according to methods which are described therein or which are known to those skilled in the art.

The example which follows describes a method of preparing a compound in accordance with the invention.

This example is not limiting and merely illustrates the present invention.

In the example of preparation of a compound of formula (I) which follows, the following definitions are used:
THF=tetrahydrofuran,
CDCl₃=chloroform-d,
LC/MS=liquid chromatography/mass spectrometry,
eq.=molar equivalent,
Mp=melting point,
s=singlet,
t=triplet,
m=multiplet,
q=quintuplet.

The ¹H and ¹³C NMR spectra were realized on two Bruker devices: AC 200 and Avance 600.

The chromatography/mass spectrometry procedures were carried out on a Micromass® “TOF” (time of flight) mass spectrometer, model LCT.

The melting points were measured on a Büchi melting point B-545 device.

### EXAMPLE

### Stage A

### Oxidation of the Alcohol Function to a Carboxylic Acid

### (Preparation of the Compounds of Formula (IV))

5.07 g of the compound of formula (V) (16.6 mmol; 1 eq.) and 166 ml of acetone, followed by 10.49 g of potassium permanganate (66.4 mmol; 4 eq.), were introduced into a 500 ml round-bottomed flask under an argon atmosphere. The synthesis of the compound of formula (V) is described in document EP 0 873 309 (preparation 10, compound (III′.1)). The reaction mixture is stirred for 3 days. The reaction medium is filtered over Celite®, rinsed with methanol and then concentrated under vacuum. The crude product (6.6 g) is purified on silica gel (150 g; 40-63 μm particles; the eluent used is a gradient: CH₂Cl₂/methanol: 90/10, in the following proportions: CH₂Cl₂/methanol/acetic acid: 89/9/2). The fractions containing the desired derivative are combined and are then concentrated under vacuum.

The proton NMR spectrum is compatible with the desired structure.

### Stage B

### Formation of the Amide Function from the Carboxylic Acid Function

### (Preparation of the Compound of Formula (III))

728 mg of carbodiimidazole (4.72 mmol; 1.1 eq.), 72 ml of anhydrous tetrahydrofuran and 1.37 g of the acid derivative of formula (IV) obtained in stage a) (1.37 mmol; 1 eq.) are introduced into a 100 ml round-bottomed flask under an argon atmosphere. After stirring for 2 hours, 0.41 ml of morpholine (413 mg; 4.72 mmol; 1.1 eq.) is added. The reaction medium is stirred for 2 hours and the mixture is subsequently washed with salt water. The aqueous phase is extracted several times with ethyl acetate. The organic phases are combined and dried over sodium sulfate, filtered and then concentrated. The crude product (1.475 g) is purified on silica gel (64 g; 40-63 μm particles; the eluant used is a mixture: cyclohexane/ethyl acetate/methanol: 45/45/10). The fractions (20 ml) containing the desired derivative are combined and concentrated under vacuum.

The ¹H and ¹³C NMR spectra are compatible with the desired structure.

The LC/MS analysis confirms the structure of the product (M+H⁺=389).

### Stage C

### Coupling Between Indolinone and Sulfonyl Chloride

### (Preparation of the Compound of Formula (I))

191 mg of the morpholine derivative of formula (III) obtained in stage b) (0.49 mmol; 1 eq.), 2 ml of dichloromethane, 11 mg of benzyltriethylammonium chloride (0.05 mmol; 0.1 eq.) and 180 mg of the sulfonyl chloride derivative of formula (II) (0.59 mmol; 1.2 eq.) are introduced into a 25 ml round-bottomed flask under an argon atmosphere. The synthesis of the compound of formula (II) is described in document EP 0 873 309 (preparation 13, reactant (2).2). After cooling of the reaction medium to −5° C. (salt water/ice bath), sodium hydroxide at 40% in water (38 mg solid NaOH; 0.95 mmol; 1.93 eq.) is added. The reaction medium is vigorously stirred at −5° C. for 2 hours and then at ambient temperature for a further 2 hours. The mixture is subsequently washed with water and the aqueous phase is extracted several times with dichloromethane. The combined organic phases are dried over magnesium sulfate, filtered, and then concentrated. The crude product (390 mg) is purified on 48 g silica gel (40-63 μm particles; the eluent used is a mixture: cyclohexane/ethyl acetate/methanol: 45/45/10). The fractions containing the desired derivative are combined and are then concentrated under vacuum.

The ¹H and ¹³C NMR spectra are compatible with the desired structure.

¹³C NMR (50 MHz in CDCl₃): 177.5; 168.4; 165.0; 157.5; 156.2; 142.9; 134.1; 132.0; 131.8; 128.2; 117.4; 114.9; 113.1; 111.7; 109.8; 68.3; 66.7; 63.9; 56.1; 52.1; 46.7; 46.1; 42.1; 31.6; 28.6; 26.0; 14.8.

¹H NMR (200 MHz in CDCl₃): 8.15 (d, 1H, J=8.16 Hz); 7.7 (d, 1H, J=8.86 Hz); 7.4 (s, 1H); 7.2 (s, 1H); 6.76 (m, 2H); 6.0 (s, 1H); 4.16 (s, 2H); 3.98 (q, 2H, 6.9 Hz); 3.8-3.4 (m, 12H); 1.75 (m, 8H); 1.5 (s, 9H); 1.4 (t, 3H).

The analysis by LC/MS confirms the structure of the desired product (M+H⁺=658).

Mp=130° C.

The compounds according to the invention were subjected to pharmacological assays which show their advantage as active substances in therapy. They were in particular tested with regard to their effects. More particularly, the affinity of the compounds of the invention for V₂ receptors was determined in an in vitro binding assay according to the technique described below.

In the following text:
EDTA=ethylenediaminetetraacetic acid,
BSA=bovine serum albumin,
AVP=vasopressin,
DMSO=dimethyl sulfoxide.

### In Vitro Affinity Measurement—Ic₅₀:

The affinity of the compounds of the invention for vasopressin V₂ receptors was measured in in vitro binding assays, as described in J. Pharmacol. Exp. Ther., (2002), 300, pp. 1122-1130.

Plasma membranes (approximately 20 μg/ml) originating from tissues or the CHO cell line expressing recombinant human vasopressin V₂ receptors are incubated for 45 minutes at 25° C. in 200 μl of TRIS-HCl buffer (50 mM; pH 8.2) containing 2 mM of MgCl₂, 1 mM of EDTA, 0.1% of BSA, 1 mg/ml of bacitracin and 3.5 nM of [H³]-AVP. The reaction is stopped by filtration and washing over GF/B filters. The nonspecific binding is determined in the presence of 1 μM of AVP. The compounds of the invention, dissolved beforehand at the concentration of 10⁻² M in DMSO, are tested in a dilution range.

For each concentration, the results are expressed as percentage inhibition of specific binding. An IC₅₀ (concentration of product that inhibits 50% of the specific binding) is determined for each of the products using the “RS1 binding” program (BBN Domain, Cambridge, Mass.).

These IC₅₀ values are generally less than 10⁻⁸ M.

The compound obtained according to the previous example of the present invention has an IC₅₀ of approximately 7.3×10⁻⁹ M.

The results of the biological assays show that the compounds have an affinity for V₂ receptors and are specific antagonists of this receptor.

The compounds according to the invention can be used for the preparation of medicaments, in particular V₂ receptor antagonist medicaments.

Thus, according to another of its aspects, a subject of the invention is medicaments which comprise at least one compound of formula (I).

Compounds that are vasopressin V₂ receptor antagonists exhibit aquaretic properties in animals and humans (Cardiovascular Drug Review, (2001), 3: pp. 201-214). Thus, the compounds according to the invention have a large range of therapeutic indications and can advantageously replace conventional diuretics in all pathologies where they are recommended in humans and in animals.

Thus, the compounds according to the invention may be of use in particular in the treatment and/or the prevention of central and peripheral nervous system conditions, cardiovascular system conditions, conditions of the endocrine and hepatic system, of the renal sphere, of the gastric, intestinal and pulmonary sphere, in opthalmology, and in sexual behavior disorders, in humans and in animals.

More particularly, the compounds according to the invention can be used in the treatment and/or the prevention of various vasopressin-dependent conditions and also in vasopressin secretion dysfunctions such as the syndrome of inappropriate ADH secretion (or SIADH), cardiovascular conditions, such as hypertension, pulmonary hypertension, cardiac insufficiency, circulatory insufficiency, myocardial infarction, atherosclerosis or coronary vasospasm, in particular in smokers, unstable angina and percutaneous transluminal coronary angioplasty (or PTCA), cardiac ischemia, disturbances in hemostasis, in particular hemophilia, Von Willebrand syndrome; central nervous system conditions, migraine, cerebral vasospasm, cerebral hemorrhage, cerebral edema, depression, anxiety, bulimia, psychotic states, memory disorders, for example; rinopathies and kidney dysfunction such as edema, renal vasospasm, necrosis of the renal cortex, nephrotic syndrome, polycystic kidney disease (or PKD) in various forms in children and in adults, hyponatremia and hypokalemia, diabetes, Schwartz-Bartter syndrome or renal lithiasis; gastric system conditions, such as gastric vasospasm, portal hypertension, hepatocirrhosis, ulcers, the pathology of vomiting, for example nausea, including nausea due to chemotherapy, travel sickness, diabetes insipidus and enuresis; hepatic system conditions such as liver cirrhosis; abdominal ascites and all disorders that cause abnormal water retention; adrenal disorders (Cushing's disease), and in particular hypercorticism and hyperaldosteronemia. The compounds according to the invention can also be used in the treatment and/or the prevention of sexual behavior disorders, in conditions of being overweight or of excess weight and obesity by advantageously replacing the conventional diuretics already used for this indication. In women, the compounds according to the invention can be used for treating dysmenorrhea or premature labor. The compounds according to the invention can also be used in the treatment of small cell lung cancers, hyponatremic encephalopathies, Raynaud's disease, pulmonary syndrome and glaucoma, and in the prevention of cataracts, in post-operative treatments, in particular after abdominal, cardiac or hemorrhagic surgery, and in treatments for disorders or diseases of the inner ear, such as Ménière's disease, tinnitus, dizziness, hearing difficulties, in particular in the low-pitch range, or buzzing in the ears, hydrops, and in particular endolymphatic hydrops.

According to another of its aspects, the present invention relates to pharmaceutical compositions comprising, as active ingredient, at least one compound according to the invention. These pharmaceutical compositions contain an effective dose of at least one compound of formula (I) according to the invention, and also at least one pharmaceutically acceptable excipient.

Said excipients are chosen, according to the pharmaceutical form and the method of administration desired, from the usual excipients that are known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or its possible salt, solvate or hydrate, can be administered in unit administration form, as a mixture with conventional pharmaceutical excipients, to animals and to human beings for the prophylaxis or the treatment of the disorders or of the diseases above.

Suitable unit administration forms include oral administration forms, such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

By way of example, a unit administration form of a compound according to the invention in tablet form may comprise the following components:

| | | |
|---|---|---|
| Compound according to the Example | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

Said unit forms contain a dose so as to allow a daily administration of from 0.5 mg to 800 mg of active ingredient per individual, more particularly from 0.5 mg to 200 mg, according to the galenic form.

There may be cases where higher or lower dosages are appropriate; such dosages do not depart from the scope of the invention. According to the usual practice, the dosage suitable for each patient is determined by the physician according to the method of administration and the weight and response of said patient.

According to another of its aspects, the present invention also relates to a method of treating and/or preventing the pathologies indicated above, which comprises the administration, to a patient, of an effective dose of a compound according to the invention, or of a hydrate or solvate thereof.

What is claimed is:

1. A compound of formula (I): 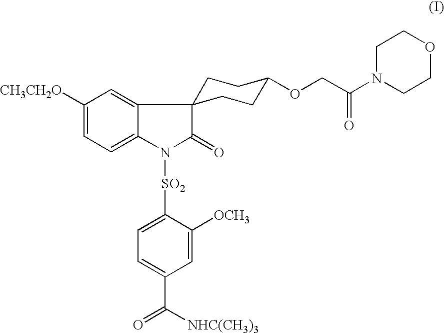
in the base, hydrate or solvate state, in the form of cis/trans isomers or of mixtures thereof.

2. A process for preparing a compound as claimed in claim 1, the process comprising reacting a morpholine derivative of formula (III) 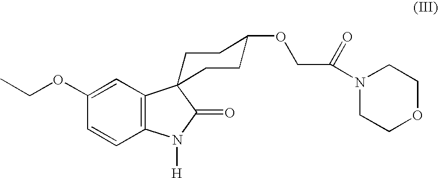
with a sulfonyl chloride of formula (II)

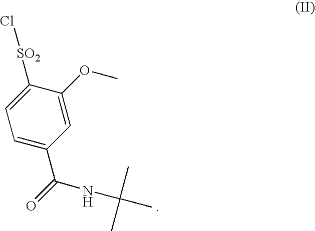

3. A pharmaceutical composition, comprising at least one compound as claimed in claim 1, and at least one pharmaceutically acceptable excipient.

4. A method of treating a patient suffering from, or subject to, a disease state selected from rinopathies and kidney dysfunction, abdominal ascites, central and peripheral nervous system conditions, cardiovascular system conditions, conditions of the endocrine and hepatic system, conditions of the renal sphere, conditions of the gastric, intestinal and pulmonary sphere, conditions in opthalmology, and conditions in sexual behavior disorders, said method comprising the step of administering an effective amount of a compound according to claim 1 to said patient.

5. A method of treating a patient suffering from, or subject to, a disease state resulting from vasopressin-dependent conditions or vasopressin secretion dysfunctions, said method comprising the step of administering an effective amount of a compound according to claim 1 to said patient.
